(19) United States
(12) Patent Application Publication
(10) Pub. No.: US 2017/0277866 A1
(43) Pub. Date: Sep. 28, 2017

(54) Method for processing of physicochemical data in order to determine legionella in water samples from a plant and execution of this method using a software application

(71) Applicants: Juan Ibañez Botella, Madrid (ES); Luis Botija Ibañez, Madrid (ES); Jaime Bouza Gonzalo, Madrid (ES); Llenalia Garcia Fernandez, Madrid (ES)

(72) Inventors: Juan Ibañez Botella, Madrid (ES); Luis Botija Ibañez, Madrid (ES); Jaime Bouza Gonzalo, Madrid (ES); Llenalia Garcia Fernandez, Madrid (ES)

(21) Appl. No.: 15/456,615

(22) Filed: Mar. 13, 2017

Related U.S. Application Data
(60) Provisional application No. 62/307,596, filed on Mar. 14, 2016.

(51) Int. Cl. G06F 19/00; G01N 33/18; G06F 17/18

(52) U.S. Cl. CPC G06F 19/707; G06F 17/18; G01N 33/18

(57) ABSTRACT

This invention relates to a method to determine proliferation risk of Legionella sp. and total aerobes, and to quantify their populations in all types of plants entailing potential proliferation and/or dissemination of these bacteria; firstly it performs previous calculations with previously measured source data in order to identify fundamental parameters for calculations. Secondly, data are sent from the user station to the central processor for processing and storage purposes. Thirdly, data are returned from the central server to the user station for storage and evaluation purposes.

### FIELD OF THE INVENTION

This invention relates to a data processing system designed to assess proliferation risk of Legionella sp. and total aerobes, and to quantify their populations in all types of plants entailing potential proliferation and/or dissemination of these bacteria and therefore increased risks for public health.

### BACKGROUND OF THE INVENTION

Legionellosis is a bacterial disease with environmental origin usually manifesting itself in two clinical forms: lung infection or Legionnaires' disease, which shows pneumonia whit high fever, and a non-pneumonic form known as Pontiac fever, which causes a mild illness with acute fever. Legionella, which causes this disease, is a type of bacteria found in the environment that can survive under a broad range of physicochemical conditions; they multiply at temperatures ranging from 20° C. (68° F.) to 45° C. (113° F.) and die at temperatures above 70° C. (158° F.), with an optimum growth range from 35° C. (95° F.) to 37° C. (98.6° F.). Their ecological niche consists of surface waters, such as lakes, rivers or ponds, from where they can colonize urban water supply systems entering the abovementioned plants through the water supply network. In this type of plants, which allow water retention and nutrient accumulation for bacteria, they multiply up to concentrations leading to human infections, as there are biofilms and favorable temperatures for their growth. The aerosol generation, which occurs in all plants listed above, enables bacteria dispersion through the air and their introduction in the respiratory system, causing the disease.

Due to such risks, these plants are monitored by health authorities, public health agencies, owners and operators. The common practices in managing plants associated to Legionellosis risks consist of several preventive and corrective maintenance tasks.

Preventive measures against Legionella proliferation often encompass a plant treatment including:

Antiscaling and anticorrosive treatment of the water in order to prevent biofilm formation. Water treatment using biocides to avoid microbiological proliferation, with daily checks of its levels. Partial renewal of the water in the plant (blowdown). Regular cleaning and disinfection of the plant.

Corrective measures are implemented when bacteria are detected (counts of Legionella or total aerobes exceed the specific threshold). These measures mainly include:
Complete water draining in the plant.
Cleaning and disinfection.
Biocide overdosing.
Total water renewal in the plant.

The early, rapid and effective detection of bacteria is critical not only to implement the corrective measures, but also to adapt the preventive measures. Nowadays, there are only analytical methods for detection; for all practical purposes, they do not provide the required information, i.e., rapid, reliable data with enough level of discrimination on the presence of live bacteria in a water sample.

The Spanish patent application P200302277 relates to a control system to prevent Legionella and other microorganisms in cooling towers. It consists of: means for determination of a substance concentration intended to prevent microorganisms in fluid samples from the towers, means for comparison of the aforementioned concentration against a specific concentration for this particular substance, first means for controlled metering of the substance, and first means of control connected to the determination means, to the comparison means and to the first metering means, so that, if the concentration identified by the determination means is lower than the specific concentration, the control means are configured to act upon the first metering means, enabling them to meter an estimated amount of the substance to the towers for microorganism prevention. The substance used for microorganism prevention is preferably a biocidal substance; for instance, the biocide may be tetrakis(hydroxymethyl)phosphonium sulfate. The means for determination of the substance concentration consist of a photometer, comprising a reservoir with intakes for fluid samples, for at least one titrant and at least a second reagent or indicator, a light-emitting diode and a light receiver within the appropriate light frequency through light filters, second means for controlled metering of an estimated amount of the titrant to the fluid sample contained in the photometer, third means for controlled metering of the second reagent or indicator (as a minimum) to the fluid sample contained in the photometer, means for stirring the mixture composed of the fluid sample, titrant and second reagent or indicator (as a minimum); thus the substance concentration for microorganism prevention in the fluid sample is determined taking into account the times that the second metering means have metered the specific amount of titrant with the purpose of allowing the mixture opacity to cut off, at a preset level, the amount of light that reaches the receiver from the emitting diode. The photometer may also include an outlet for fluid samples, for calibrating the volume of the mixture to be analyzed.

Both the titrant and the second reagent or indicator (as a minimum) will depend on the substance used for microorganism prevention, since the titration method varies from substance to substance. For example, if the biocide is tetrakis(hydroxymethyl)phosphonium sulfate, the titrant may be potassium iodide and the second reagents may be starch and selective catalytic salts.

The main issues of these methods are listed below:

Long time needed to obtain results (from several hours to 15 days from sample reception, depending on the analytical technique).
Rapid tests (hours) are carried out using PCR techniques for genetic material analysis and do not offer an accurate distinction between live or dead bacteria; this distinction is essential in identifying proliferation and dispersion risks of Legionella and, subsequently, public health risks.
Obtaining reliable data entails high costs related to analyses, and these costs increase proportionally to the increment of the required or desired frequency for availability of the microbiological information.

This invention provides real time, quantitative and qualitative estimates of the presence of aerobic bacteria and particularly Legionella sp. using a mathematical model with high goodness of fit and predictive accuracy, both improved as the invention is used thanks to a machine learning system; this is based on several water physicochemical parameters that can be easily and quickly measured even by automatic means, using measuring equipment and/or systems generally available in the market. This capacity allows plant managers to know in advance the risk of Legionella proliferation at their plant. Thus they can decide the type and scope of the appropriate preventive and/or corrective measures for the plant at a specific point in time, or simply track their maintenance and operation plan with anticipated control.

### DESCRIPTION OF THE DRAWINGS

With the aim of complementing the present description and contributing to a better understanding of the invention characteristics, according to a preferable example of the invention embodiment, a set of drawings is attached to this description as an integral part of it, including the following information by way of illustration and not limitation:

FIG. 1 is a flow diagram representing the information exchange between the user station (3) and the central server (6) using source data, and showing the following elements:

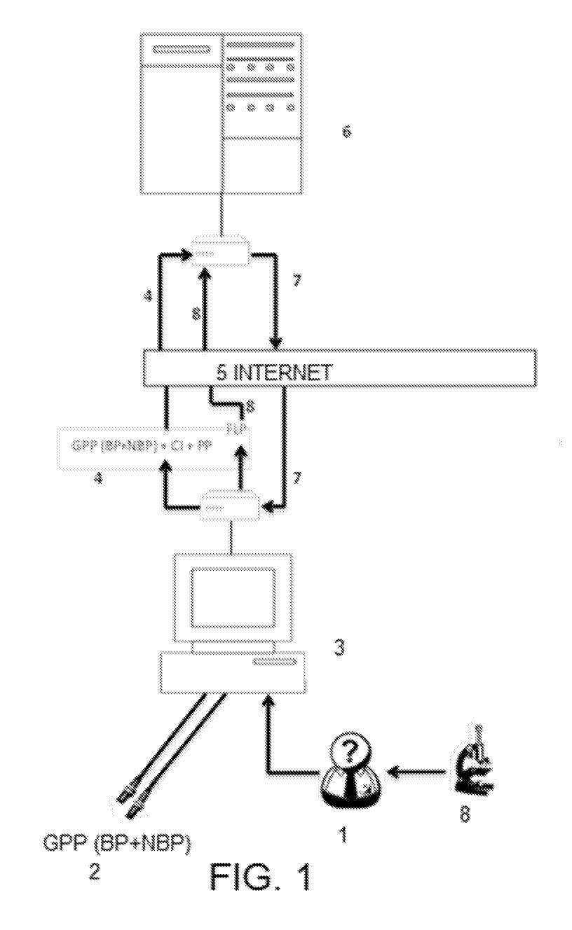

1. User

2. General physicochemical parameters (GPP)

3. User station

4. Parameters required for diagnostic purposes (GPP+CI+PP)

5. The Internet

6. Central server

7. Processing results

8. Feedback and learning parameter (FLP)

FIG. 2 is a flow diagram representing the information exchange between the user station (3) and the central server (6), with periodic feedback and learning parameter (FLP) data (8) from laboratory analyses.

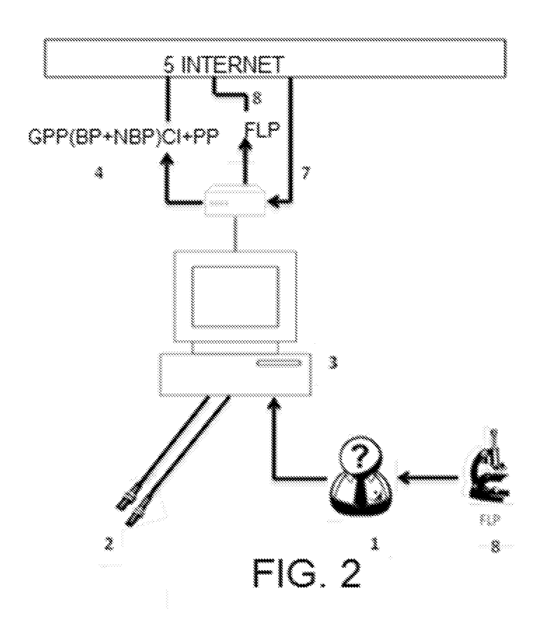

### DESCRIPTION OF THE INVENTION

This computer-assisted method is aimed to process the physicochemical data of the water, manually collected or automatically collected through rapid analysis systems and/or equipment, providing the risk of microbiological presence (Legionella sp. and total aerobes), as well as a numerical estimation of the corresponding population.

In automatic mode, the user station (3) reads a calibrated analog signal, obtained from the measurement equipment, of the required physicochemical parameters, recording the relevant data for their analysis and processing. In manual mode, the user (1) manually enters data through a user interface.

The parameters used for both analysis and machine learning are classified as follows:

There are two types of parameters for analyses: general physicochemical parameters and basic parameters.

General physicochemical parameters (GPP): They refer to parameters that generally participate in the process used to provide diagnoses.
Temperature (T).
Calcium hardness (CH).
Magnesium hardness (MH).
Total dissolved solids (TDS).
Turbidity (TURB).
pH.
Conductivity (COND).
Iron (Fe).
Total hardness (TH).
Total alkalinity (CAT).
Simple alkalinity (TA).
Chlorides (Cl—).
Sulfates (SO₄⁻²).
Bicarbonates (HCO₃⁻).
Carbonates (CO₃⁻²).

Basic parameters (BP): They refer to GPP featuring indispensable values for achieving diagnoses with the highest level of accuracy; the model itself cannot calculate their value. Basic parameters are:
Total alkalinity (CAT).
Calcium hardness (CH).
pH.
Total dissolved solids (TDS).
Conductivity (COND).
Temperature (T).

There are also non-basic parameters (NBP), and feedback and learning parameters (FLP).

The process allows the calculation of these parameters according to the data from the “basic parameters”, hence the lack of these parameters will not prevent the processing and efficient calculation of diagnoses; however, in some cases this lack may affect the predictive accuracy and bring a loss of efficiency for diagnoses.

Feedback and learning parameters (FLP) of the system (8): They include GPP and quantification of live bacteria (Legionella sp. and total aerobes). The FLP should be measured at a laboratory on a single water sample from the plant.

### Preferable Embodiment of the Invention

The present preferable embodiment of the invention refers to a method that firstly performs previous calculations with previously measured source data in order to identify fundamental parameters for calculations. Secondly, data are sent from the user station to the central processor for processing and storage purposes. Thirdly, data are returned from the central server to the user station for storage and evaluation purposes.

Previous calculations may be manually obtained or may be implemented through the user's computer; in any case certain previous calculations must be executed in order to obtain several calculated indices (CI) based on either automatically entered data or manually entered data through the user's computer interface.

Upon calculation of such indices, their values will be added to those of the parameters required for diagnostic purposes (4):

The indices to be determined are:

Langelier saturation index (LSI), which can be calculated from the following equation: LSI=pH−pHsat, where pHsat is determined from the equation:

pHsat=(9.3+A+B)−(C+D), where A= 1/10(log[TDS]−1), B=−13.12 log[T(° C.)+273.2]+34.55, C=log[CH]−0.4 and D=log CAT.

Ryznar stability index (RSI), which can be calculated from the following equation:

RSI=2(pHsat)−pH, where pHsat=(9.3+A+B)−(C+D) and where A= 1/10(log[TDS]−1), B=−13.12 log[T(° C.)+273.2]+34.55, C=log [CH]−0.4 and D=log CAT.

Puckorius scaling index (PSI), which can be calculated from the following equation: PSI=2(pHsat)−pHeq, where pHsat=(9.3+A+B)−(C+D) and where A= 1/10(log [TDS]−1), B=−13.12 log[T(° C.)+273.2]+34.55, C=log [CH]−0.4, D=log CAT, and pHeq=1.465(log [CAT])+4.54.

Likewise several parameters from the plant itself (PP) must be added to the water parameter list.

Age of the plant (date of analysis—date of plant commissioning), water volume in the circuit, temperature difference in the plant and plant's power.

Once the source data are collected, and the calculated data and plant parameters are added, this data set is sent via the Internet (5) to a central server (6), where it is processed using the automatic actions listed below:
1. Scrubbing and cleansing of the entered data: After entering the data into the system, statistical tools for detection of outliers and abnormal data are executed for the purpose of correcting systematic or user-entered errors.
2. Classification: It is executed using a statistical model of cluster organization that defines the inner correlation structure of the data to be analyzed, allocating them to a cloud data cluster for which they are homogeneous. Defining a data cluster by mathematical calculation in respect whereof the sample to be analyzed is homogeneous enables the improvement of the goodness of fit in the predictive models for Legionella and aerobes described below.
3. Legionella prediction: Upon defining the data cluster structure, two mathematical models will be executed: one of them provides an estimated quantification for Legionella, while the other predicts the risk of presence of Legionella according to the database physicochemical parameters. Predictions for Legionella quantification are obtained by a mixed linear regression model, identifying the implicit clustering levels of data as random effects. Risk prediction for Legionella presence is achieved through a logistic regression model used to calculate Legionella probabilities according to the physicochemical parameters. The models are verified using the goodness of fit and accuracy parameters of the resulting prediction.
4. Aerobe prediction: At the same time, the system executes two additional mathematical models that predict aerobe quantification and risk of presence of aerobes, with the “presence of aerobes” based on a user-defined quantification of colony forming units. Both statistical techniques use mixed regression models: a linear model for quantification and a logistic model for the existence of risk. The random effects entered in the model are collected using the precalculated clustering structure, which is “optimum” for goodness of fit improvement.
5. Results: Analysis results will be sent through the Internet (5) from the central computer (6) to the user's computer (3) or mobile device, appearing in its interface. Using this interface, users can download the analysis results as electronic reports.
6. Result storage: The obtained results are kept both in the central server database (6) and user's computer database (3) for future reference when needed.

On a regular basis, with a user-defined frequency according to specific needs, interests or duties, the system will receive FLP data (8) from laboratory analyses. These data are entered through the user interface and automatically sent via the Internet (5) to the central server (6), where the following automatic actions are executed:
1. Validation of the entered data: After entering the data into the system, statistical tools for detection of outliers and abnormal data are executed for the purpose of correcting systematic or user-entered errors.
2. Incorporation into databases: The individualized data entered in the system are incorporated into the existing database, modifying and perfecting the statistical model.
3. Cluster reorganization: On a regular basis, with an adjustable frequency, an automatic revision of the cluster structure is performed, estimating again the aforementioned structure of correlation. The expansion of the database size as the system is used together with the automatic reorganization of clusters will provide a constant improvement in relation to the goodness of fit in predictive models and the definition of the inherent data structure. As a result of this process, the existing number of clusters can be kept or changed.
4. Automation and improvement of predictive models: The cluster structure is automatically added to the predictive models, progressively improving the goodness of fit for risk and quantification analyses, expanding the model capacity to obtain a higher level of accuracy in the reported estimates, and improving the estimates even when data are more heterogeneous and variable.

The method for information exchange between a user station and the central server follows the protocol below:

The user (1) automatically or manually enters the GPP (2) in the desktop application of its user station or PC (3). The user station (3), with dynamic IP, communicates through the Internet (5) with the central server (6) by invoking its IP number (static IP). When the secure communication channel is established between the user station (3) and the central server (6), the information flow may be bidirectional. Since user stations (3) have dynamic (changeable) IPs and the server (6) has a static (unchangeable) IP, communication will always be established by the user stations (3). Once the user (1) has entered the general physicochemical parameters (GPP) (2) in the user station (3), the desktop application estimates the calculated indices (CI) and adds them, together with the plant parameters (PP), to the GPP (2). Then this set of GPP+Cl+PP data (4) is sent to the central server (6) through the secure channel created on the Internet (5). The central server (6) receives the set of GPP+CI+PP data (4) and processes it by executing the scrubbing, cleansing, classification, Legionella prediction and aerobe prediction. Once the processing results (7) are obtained, the central server (6) stores them in a database and sends them through the secure channel created on the Internet (5) to the user station (3), where they will be presented to the user (1) and stored in a local database.

When the information exchange is complete, the secure communication channel between the user station (3) and the server (6) is closed.

The user (1) will receive the FLP (8) from a certified laboratory, at a frequency determined at user's discretion or according to the requirements of the applicable law or quality standards to which the plant is subject, and will enter them in the user station (3). As explained above, the user station (3) will establish a secure communication channel with the central server (6) trough the Internet (5) and will send the FLP (8). Upon reception of the FLP (8), the central server will proceed with the validation and cleansing. Afterwards, it will include them in the central database for the subsequent cluster reorganization, and the revision and improvement of predictive models.

1- A method of physicochemical data processing for determination of Legionella bacteria in water samples, comprising the following stages:
a- obtaining general physicochemical parameters (GPP) (2) from source data
b- determination of calculated indices (CI) based on the general physicochemical parameters (GPP) (2), where the calculated indices (CI) include: Langelier saturation index (LSI), Ryznar stability index (RSI) and Puckorius scaling index (PSI)
c- determination of the plant parameters (PP), these parameters comprising: age of the plant, being the difference between the date of analysis and the date of plant commissioning, water volume in the circuit, temperature difference in the plant and plant's power
d- data submission from stages a, b and c to the central server (6) via the Internet (5)
e- processing of the data from the aforementioned stages a, b and c, which includes: scrubbing and cleansing of input data, data classification, Legionella prediction and aerobe prediction
f- submission of the results of stage e from the central server (6) to the user station (3) through the Internet (5).
g- Storage of data from stage e, both in the central server (6) and the user station (3).

2- The method of claim 1, wherein the general physicochemical parameters (GPP) (2) obtained from the source data are collected by the computer (3) by means of a calibrated analog signal coming from the measuring equipment of the required physicochemical parameters.

3- The method of claims 1 and 2, wherein the general physicochemical parameters (GPP) (2) obtained from the source data include temperature (T), calcium hardness (CH), magnesium hardness (MH), total dissolved solids (TDS), turbidity (TURB), pH, conductivity (COND), iron (Fe), total hardness (TH), total alkalinity (CAT), simple alkalinity (TA), chlorides (Cl⁻), sulfates (SO₄⁻²), bicarbonates (HCO₃⁻) and carbonates (CO₃⁻²).

4- The method of claim 1, wherein the Langelier saturation index (LSI) is calculated from the following equation: LSI=pH−pHsat, where pHsat is determined from the equation:
pHsat=(9.3+A+B)−(C+D), where A= 1/10(log[TDS]−1), B=−13.12 log [T(° C.)+273.2]+34.55, C=log [CH]−0.4 and D=log CAT.

5- The method of claim 1, wherein the Ryznar stability index (RSI) is calculated from the following equation: RSI=2(pHsat)−pH, where pHsat=(9.3+A+B)−(C+D) and where A= 1/10(log[TDS]−1), B=−13.12 log [T(° C.)+273.2]+34.55, C=log[CH]−0.4 and D=log CAT.

6- The method of claim 1, wherein the Puckorius scaling index (RSI) is calculated from the following equation: PSI=2(pHsat)−pHeq, where pHsat=(9.3+A+B)−(C+D) and where A= 1/10(log[TDS]−1), B=−13.12 log [T(° C.)+273.2]+34.55, C=log [CH]−0.4, D=log CAT, pHeq=1.465(log[CAT])+4.54.

7- The method of claim 1, wherein the scrubbing and cleansing of data from stage e is executed using statistical tools for detection of outliers and abnormal data, correcting systematic or user-entered errors.

8- The method of claim 1, wherein the classification of data from stage e is executed using a statistical model of cluster organization that defines the inner correlation structure of the data to be analyzed, allocating them to a cloud data cluster for which they are homogeneous.

9- The method of claim 1, wherein the Legionella prediction is determined by means of two mathematical models that estimate a Legionella quantification and predict the risk of presence of Legionella according to the physicochemical data from stages a, b and c.

10- The method of claim 9, wherein Legionella quantification is obtained by a mixed linear regression model, identifying the implicit clustering levels of data as random effects.

11- The method of claim 9, wherein risk prediction for Legionella presence is achieved through a logistic regression model used to calculate Legionella probabilities according to the general physicochemical parameters (GPP) from stages a, b and c.

12- The method of claim 1, wherein aerobe prediction from the stage e is executed using two mathematical models that predict aerobe quantification and existence of risk of aerobes.

13- The method of claim 12, wherein aerobe quantification is obtained by linear regression.

14- The method of claim 12, wherein the existence of risk is determined using a logistic regression model.

15- The method of claim 1, wherein the central server (6) will periodically receive FLP source data (8) from laboratory analyses, which are automatically sent from the user interface (3) via the Internet (5) to the central server (6).

16- The method of claim 15, wherein upon data entry in the central server (6), statistical tools for detection and correction of outliers and abnormal data due to systemic or user-entered errors are executed.

17- The method of claims 15 and 16, wherein the individualized input data of the server (6) are incorporated into the existing database.

18- The method of claims 15-17, wherein an automatic revision of the cluster structure is periodically implemented, estimating again the aforementioned structure of correlation.

19- The method of claim 18, wherein the cluster structure is automatically added to the predictive models.

20- A method of information exchange for Legionella determination in water samples using a software application of a user station or PC (3), with dynamic IP, and a central server (6) through the Internet (5) by invoking its IP number, comprising the following stages:
the general physicochemical parameters GPP (2) from source data are entered into the user station (3)
the application, which is set up in the user station (3), estimates the calculated indices (CI) and adds them, together with the plant parameters (PP), to the GPP (2)
this data set (4) is sent by the user station to the central server (6) through the secure channel created on the Internet (5)
the central server (6) receives the set of GPP+CI+PP data (4) and processes it by executing the scrubbing, cleansing, classification, Legionella prediction and aerobe prediction
once the processing results (7) are obtained, the central server (6) stores them in a database and sends them through the secure channel created on the Internet (5) to the user station (3), where they will be presented to the user (1) and stored in a local database
when the information exchange is complete, the secure communication channel between the user (3) and the server (6) is closed.
